(19) United States
(12) Patent Application Publication
(10) Pub. No.: US 2025/0135055 A1
(43) Pub. Date: May 1, 2025

(54) MANUFACTURING METHOD OF LIGHT-TRIGGERED LIGHT-TRANSMITTING CLEANING STRUCTURE AND CLEANING METHOD USING LIGHT-TRIGGERED LIGHT-TRANSMITTING CLEANING STRUCTURE

(71) Applicant: Geeder company Limited, Taichung City (TW)

(72) Inventors: CHENG-SHENG TSUNG, Taichung City (TW); CHUN-CHIH CHIU, Taichung City (TW); YUAN-QIU ZHENG, Taichung City (TW)

(21) Appl. No.: 18/383,914

(22) Filed: Oct. 26, 2023

(51) Int. Cl. A61L 2/10

(52) U.S. Cl. CPC A61L 2/10

(57) ABSTRACT

The present invention provides a manufacturing method and cleaning method of light-triggered light-transmitting cleaning structure. The manufacturing method includes following steps: step S1, providing a light-transmitting substrate; step S2, forming a nanoparticle layer on the substrate surface; step S3, heating the light-transmitting substrate to a softening temperature and allowing metallic nanoparticles to permeate the light-transmitting substrate; step S4, cooling for the metallic nanoparticles to form a doped structure in the light-transmitting substrate, forming the light-triggered light-transmitting cleaning structure. The cleaning method forms an optical Tamm state by irradiating said structure with a light source to resonate, such that the optical Tamm state interacts with ambient substance to form a cleaning substance for removing pollutants. The present invention achieves the purpose of pollutants cleaning under the conditions of high transparency, high light transmission, and long service life.

### BACKGROUND OF THE INVENTION

### 1. Field of the Invention

The present invention relates to a manufacturing method and a cleaning method, and more particularly, to a manufacturing method of light-triggered light-transmitting cleaning structure and cleaning method using light-triggered light-transmitting cleaning structure.

### 2. Description of the Related Art

The traditional bacteriostatic, antibacterial, or sterilizing glass is usually coated with a layer of sterilizing material (such as silver, copper, zinc, and other metals with sterilizing function) on the surface of the glass, thereby achieving the sterilizing effect by use of the sterilizing material capable of destroying viruses or bacteria.

However, the sterilizing material covering the surface of the glass usually has its own color (for example, silver is slightly yellowish, and zinc presents a blue-silver color), not only reducing the transmittance of the glass, but also causing a color difference, failing to meet the usage demand. Furthermore, the sterilizing material coated on the glass surface will usually suffer from loss, wearing or peeling after a long-term of usage, which lead to a significant deterioration of the sterilizing ability of the glass.

Also, the manufacturing process of the aforementioned glass has high demands for environmental and process cleanliness, and the manufacturing procedure is relatively complicated, so that the aforementioned glass also has the issue of high manufacturing cost.

### SUMMARY OF THE INVENTION

The present invention aims at resolving the issue of transmittance and color difference of the conventional coated sterilizing glass, and also improving the issue of the coating membrane having a reduced service life due to contact wearing and prolonged light exposure, resulting in a reduced sterilizing ability.

For achieving the aforementioned objectives, the present invention provides a manufacturing method of light-triggered light-transmitting cleaning structure, comprising following steps: step S1, providing a light-transmitting substrate having a surface; step S2, forming a nanoparticle layer having a plurality of metallic nanoparticles on the surface; step S3, heating the light-transmitting substrate to a softening temperature and keep heating for a heating time allowing the light-transmitting substrate to enter a softened status, so that the metallic nanoparticles permeate the light-transmitting substrate; and step S4, cooling the light-transmitting substrate doped with the metallic nanoparticles to a room temperature for the metallic nanoparticles to form a doped structure in the light-transmitting substrate, thereby forming the light-triggered light-transmitting cleaning structure.

In an embodiment of the present invention, the light-transmitting substrate is formed of an insulating material.

In an embodiment of the present invention, the light-transmitting substrate is formed of glass.

In an embodiment of the present invention, the metallic nanoparticles are nanometer-sized metal materials or metal oxide materials.

In an embodiment of the present invention, the softening temperature is the softening point temperature of the glass.

In an embodiment of the present invention, the heating time ranges from 3 to 20 minutes.

In an embodiment of the present invention, the light-transmitting substrate is formed of an amorphous polymer.

In an embodiment of the present invention, the metallic nanoparticles are nanometer-sized metal oxide materials.

In an embodiment of the present invention, the softening temperature is between the glass transition temperature (Tg) and the viscous flow temperature (Tf) of the amorphous polymer.

In an embodiment of the present invention, in step S4, the metallic nanoparticles form a grain boundary themselves or with the ambient substance molecules, and the metallic nanoparticles are combined with the grain boundary to form the doped structure.

Also, the present invention provides a cleaning method using the light-triggered light-transmitting cleaning structure, comprising following steps: step S5, irradiating the light-triggered light-transmitting cleaning structure with a light source, such that the light source causes a surface plasmon polariton to be formed on a surface of the metallic nanoparticles of the doped structure, and a Tamm plasmon polariton is formed at the grain boundary of the doped structure, whereby the surface plasmon polariton and the Tamm plasmon polariton resonate with each other to form an optical Tamm state; and Step S6, performing an interactive oscillation between the optical Tamm state and ambient substances of the light-triggered light-transmitting cleaning structure to form a cleaning substance, which spreads outward from a periphery of the light-triggered light-transmitting cleaning structure to remove a pollutant around the light-triggered light-transmitting cleaning structure.

In an embodiment of the present invention, the wavelength of the light source ranges from 100 to 1000 nanometers.

In an embodiment of the present invention, when the light-transmitting substrate is formed of glass, the wavelength of the light source ranges from 320 to 570 nanometers.

With such configuration, the doped structure in the light-transmitting structure manufactured by the present invention is transparent, and the cleaning substance generated by the doped structure spreads toward the surrounding space of the light-transmitting structure. Therefore, the aforementioned light-transmitting structure maintains the transmittance and improves the sterilizing capability.

### BRIEF DESCRIPTION OF THE DRAWINGS

FIG. 1 is a flow chart of the manufacturing method of light-triggered light-transmitting cleaning structure in accordance with an embodiment of the present invention.

FIG. 2 is a flow chart of the cleaning method of light-triggered light-transmitting cleaning structure in accordance with an embodiment of the present invention.

FIG. 3 is a schematic view of a practical operation step in accordance with an embodiment of the present invention, illustrating a nanoparticle layer formed on the surface of the light-transmitting substrate.

FIG. 4 is a schematic view of a practical operation step in accordance with an embodiment of the present invention, illustrating a plurality of metallic nanoparticles at the nanoparticle layer permeating the light-transmitting substrate.

FIG. 5 is a schematic view of a practical operation step in accordance with an embodiment of the present invention, illustrating the light-triggered light-transmitting cleaning structure having a doped structure.

### DETAILED DESCRIPTION OF THE INVENTION

The aforementioned and further advantages and features of the present invention will be understood by reference to the description of the preferred embodiment in conjunction with the accompanying drawings where the components are illustrated based on a proportion for explanation but not subject to the actual component proportion.

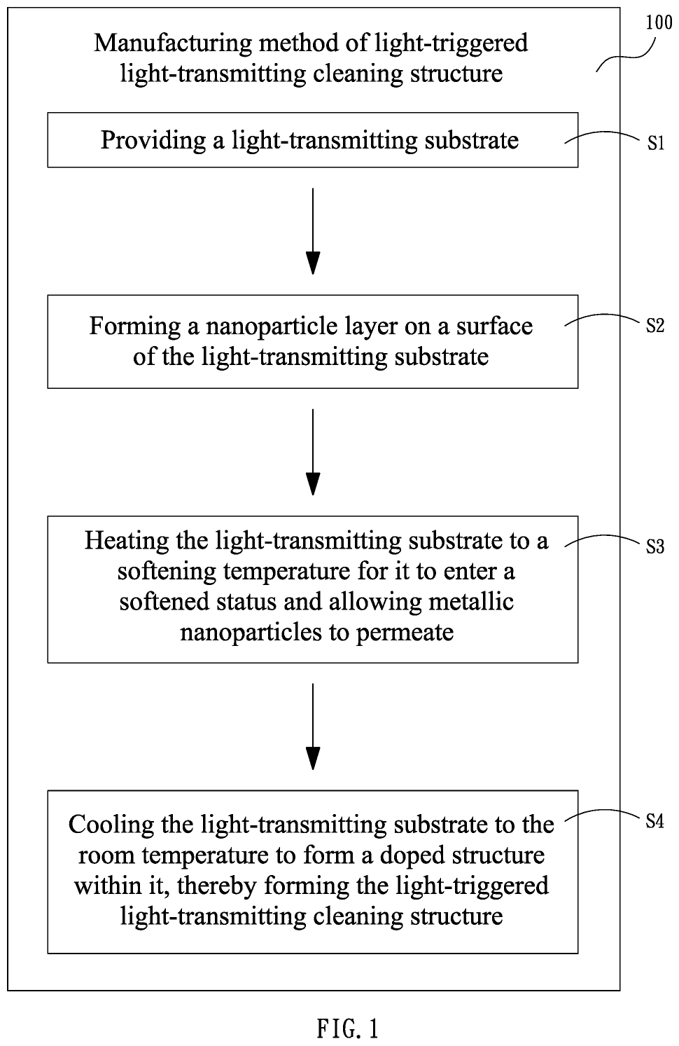

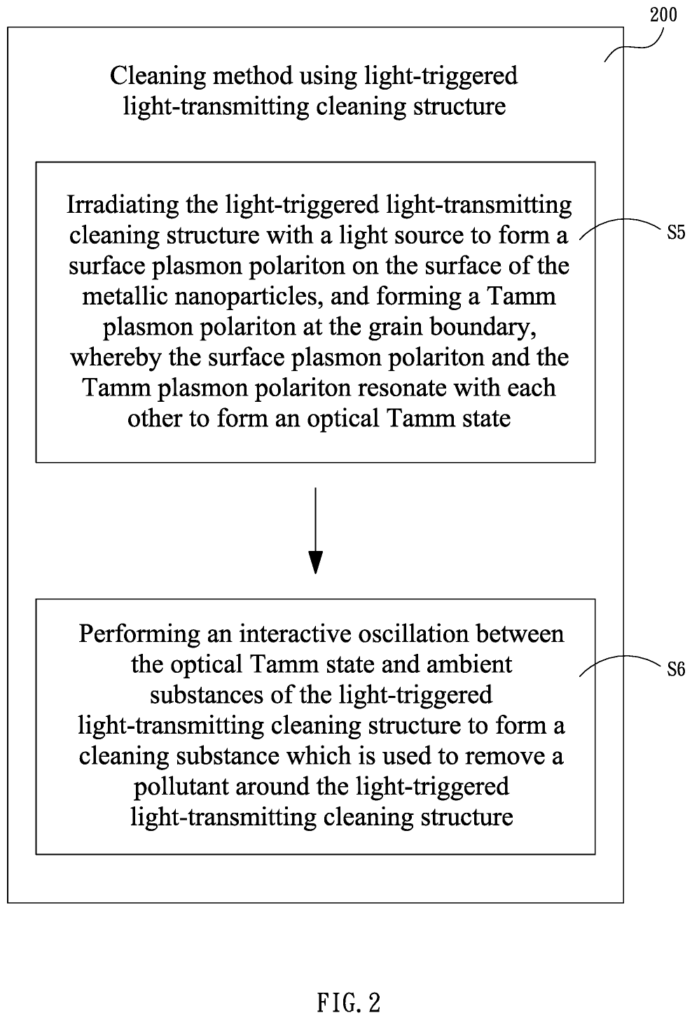

Referring to FIG. 1 to FIG. 5, a manufacturing method 100 of a light-triggered light-transmitting cleaning structure 1 is disclosed, comprising steps S1 to S4.

In step S1, a light-transmitting substrate 10 is provided, wherein the light-transmitting substrate 10 comprises a surface 11. Therein, in the embodiment, the light-transmitting substrate 10 is formed of an insulating material selected from glass or amorphous polymer. Glass is mainly formed of silicon oxide. However, the present invention does not exclude the use of other transparent oxide material. The amorphous polymer is allowed to be polyethylene (PE), polystyrene (PS), polyvinyl chloride (PVC), polyethylene terephthalate (PET), polybutylene terephthalate (PBT), acrylonitrile-butadiene-styrene copolymer (ABS), polymethyl methacrylate (PMMA), polyetherimide (PEI), polyphenylene sulfide (PPS), polyvinylidene fluoride (PVDF), polytetrafluoroethylene (PTFE), polyether sulphone (PES), polyamide resin, styrene-acrylonitrile copolymer (AS), polypropylene (PP), polyvinyl fluoride (PVF), polyvinylidene chloride (PVDC), polysulfone (PSF), polyphenylene oxide (PPO), polycarbonate (PC), chlorinated polyether, polyarylate (PAR), polyetheretherketone (PEEK), polyimide (PI), epoxy resin (EP), phenol formaldehyde resin (PF), polyphenyl ester, polybenzimidazole (PBI), polyamide-imide (PAI), etc. Also, the transmittance of the amorphous polymer is at least 50%.

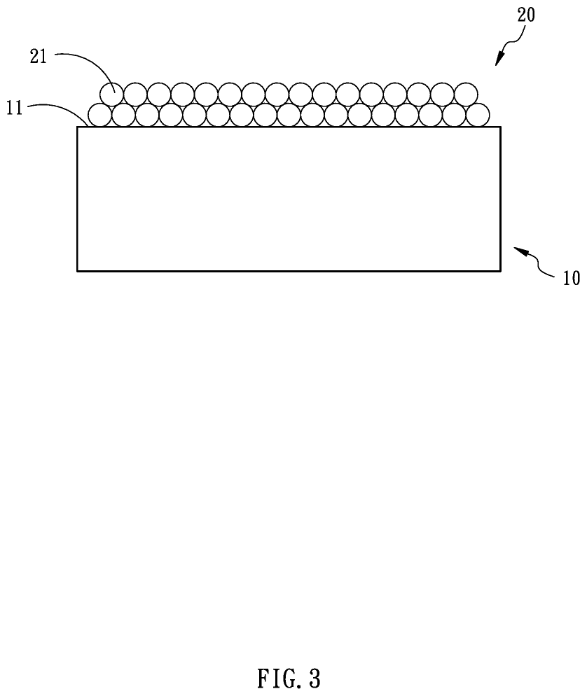

In step S2, a nanoparticle layer 20 is formed on the surface 11 of the light-transmitting substrate 10. The nanoparticle layer 20 comprises a plurality of metallic nanoparticles 21 (as shown by FIG. 3). Therein, in the embodiment, when the light-transmitting substrate 10 is a glass, the metallic nanoparticles 21 are allowed to be nanometer-sized metal materials or metal oxide materials. When the light-transmitting substrate 10 is an amorphous polymer, the metallic nanoparticles 21 are nanometer-sized metal oxide materials. The metal material is selected from metals such as gold, silver, copper, zirconium, aluminum, lithium, nickel, and zinc. The metal oxide material is selected from transparent conductive metal oxides such as indium oxide, indium tin oxide, indium gallium zinc oxide, fluorine-doped tin oxide, and silicon-doped zinc oxide.

Also, when the light-transmitting substrate 10 is a glass, since glass has higher heat resistance and scratch resistance, the nanoparticle layer 20 is allowed to be formed on the light-transmitting substrate 10 through evaporation, sputtering, chemical vapor deposition, coating, scrape coating, or roller coating. When the light-transmitting substrate 10 is an amorphous polymer, since the amorphous polymers generally have lower heat resistance and scratch resistance, the nanoparticle layer 20 is preferably formed on the light-transmitting substrate 10 through coating, spraying, scrape coating, or roller coating; also, before the forming step of the nanoparticle layer 20, the light-transmitting substrate 10 is allowed to be coated with an additive (such as organic solvents like acetone or toluene), such that the additive improves the adhesion between the nanoparticle layer 20 and the light-transmitting substrate 10.

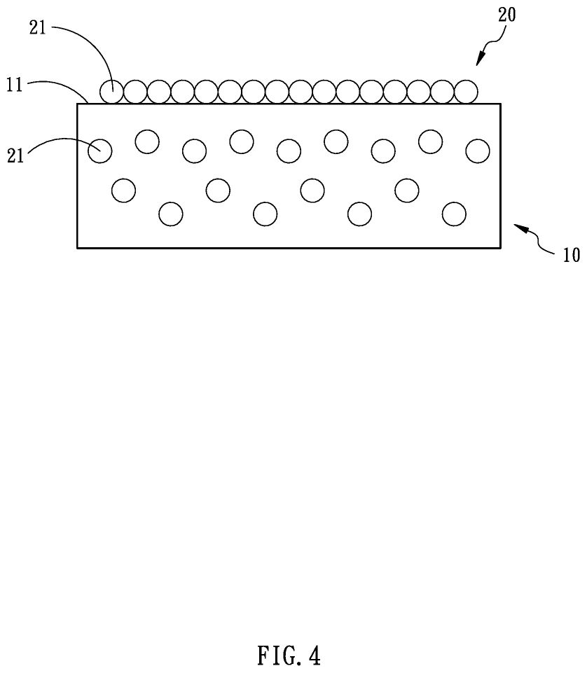

In step S3, the light-transmitting substrate 10 is heated to a softening temperature and continuously heated for a heating time allowing the light-transmitting substrate 10 to enter a softened status, so that the metallic nanoparticles 21 permeate the light-transmitting substrate 10 (as shown by FIG. 4). Therein, in the embodiment, when the light-transmitting substrate 10 is a glass, the softening temperature is the softening point temperature of glass (approximately 650 degrees Celsius to 750 degrees Celsius).

When the light-transmitting substrate 10 is an amorphous polymer, the softening temperature is between the glass transition temperature (Tg) and the viscous flow temperature (Tf) of the amorphous polymer. When the light-transmitting substrate 10 is a glass, the heating time ranges from 3 minutes to 20 minutes.

Also, when the light-transmitting substrate 10 is in the high temperature softening status, the molecular motion in the light-transmitting substrate 10 is intensified due to the high temperature, and the permeating speed of the metallic nanoparticles 21 is improved due to the intensified molecular motion. And, during the permeating process of the metallic nanoparticles 21, the nanoparticle layer 20 gradually disappears.

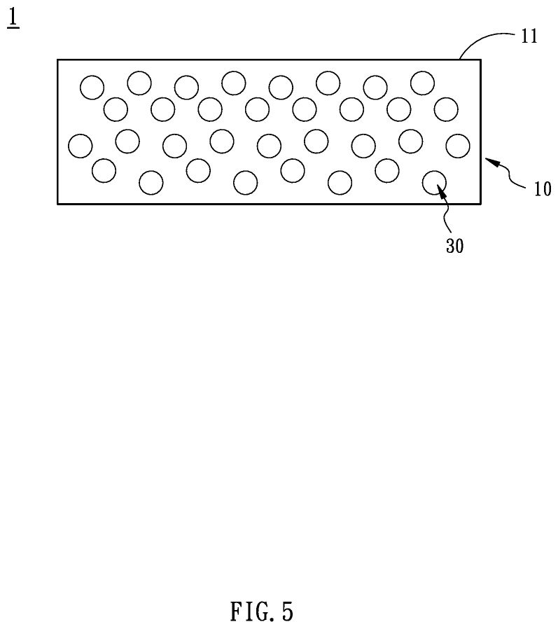

In step S4, the light-transmitting substrate 10 doped with the metallic nanoparticles 21 is cooled down to the room temperature for the metallic nanoparticles 21 to form a doped structure 30 in the light-transmitting substrate 10, thereby forming the light-triggered light-transmitting cleaning structure 1 (as shown by FIG. 5). Therein, in the embodiment, the metallic nanoparticles 21 form a grain boundary themselves or with the ambient substance molecules, and the metallic nanoparticles 21 are combined with the grain boundary to form the doped structure 30. For further illustrations, the grain boundary is allowed to be formed at the metallic nanoparticles 21 themselves, at the joint boundary around the metallic nanoparticles 21, or at the creased deformation position of the ambient substance molecules doped with and compressed by the metallic nanoparticles 21.

The present invention further provides a cleaning method 200 using the light-triggered light-transmitting cleaning structure 1, comprising following steps S5 to S6.

In step S5, the light-triggered light-transmitting cleaning structure 1 is irradiated with a light source, such that the light source causes a surface plasmon polariton (SPP) to be formed on a surface of the metallic nanoparticles 21 of a doped structure 30, and a Tamm plasmon polariton (TPP) is formed at the grain boundary of the doped structure 30, whereby the surface plasmon polariton and the Tamm plasmon polariton resonate with each other to form an optical Tamm state (OTS).

Therein, the wavelength of the light source ranges from 100 to 1000 nanometers. In another preferred embodiment, when the light-transmitting substrate 10 is formed of glass, the wavelength of the light source ranges from 320 to 570 nanometers, and preferably ranges from 350 to 450 nanometers. The light source of such purple-based light improves the generation efficiency of the surface plasmon polariton and the Tamm plasmon polariton.

In step S6, an interactive oscillation is performed between the optical Tamm state and ambient substances of the light-triggered light-transmitting cleaning structure 1 to form a cleaning substance, which spreads outward from a periphery of the light-triggered light-transmitting cleaning structure 1 to remove a pollutant around the light-triggered light-transmitting cleaning structure 1. Therein, for example, the ambient substance is moisture, and the cleaning substance is the hydrogen ions (H⁺) and hydroxide ions (OH) dissociated by the interactive oscillation between the moisture and the optical Tamm state, such that the cleaning substance removes the pollutant on the surface of the light-triggered light-transmitting cleaning structure 1. Notably, the dissociated hydrogen ions (H⁺) and hydroxide ions (OH⁻), instead of only staying on the surface of the light-triggered light-transmitting cleaning structure 1, would spread into the air by diffusion or floating to achieve the purpose of removing the pollutants. For another example, the ambient substance is allowed to be oxygen, and the cleaning substance is the superoxide molecules (.O₂⁻) and oxygen ions (O²⁻) obtained by the interactive oscillation between the oxygen and the optical Tamm state.

Further, if said pollutant is virus, bacterium or mold, said cleaning substance is able to directly destroy said pollutant from the surrounding environment of the light-triggered light-transmitting clean structure 1. For example, the hydrogen ions (H⁺) and the hydroxide ions (OH⁻) are capable of destroying bacteria or viruses. If said pollutant is a suspended particulate matter, such as PM10, PM2.5 or haze, the said cleaning substance is capable of capturing said pollutant and causing said pollutant to gather and settle on the ground, thereby reducing the probability of said pollutant accumulating on the surface of the light-triggered light-transmitting cleaning structure 1.

With the foregoing configuration, the present invention achieves following advantages.

The doped structure 30 of the light-triggered light-transmitting cleaning structure 1 is colorless and transparent. Therefore, the light-triggered light-transmitting cleaning structure 1 is highly transparent and has a high transmittance, without causing a color difference.

The metallic nanoparticles 20 applied by the present invention are not limited to the metals having a sterilizing function. In other words, ordinary metals are also applicable. Therefore, the present invention has high flexibility, and the utility cost is relatively low.

The doped structure 30 of the light-triggered light-transmitting cleaning structure 1 manufactured by the present invention is formed inside the structure thereof, instead of being exposed out of the structure. Therefore, the present invention is prevented from issues of wearing and deterioration of sterilizing capability after a long-term usage.

The cleaning substance generated by light irradiation of the present invention spreads out from the periphery of the light-triggered light-transmitting cleaning structure 1, so as to effectively increase the overall cleaning range and improve the overall sterilizing capability.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A manufacturing method of light-triggered light-transmitting cleaning structure, comprising following steps:
step S1: providing a light-transmitting substrate having a surface;
step S2: forming a nanoparticle layer having a plurality of metallic nanoparticles on the surface;
step S3: heating the light-transmitting substrate to a softening temperature and keep heating for a heating time allowing the light-transmitting substrate to enter a softened status, so that the metallic nanoparticles permeate the light-transmitting substrate; and
step S4: cooling the light-transmitting substrate doped with the metallic nanoparticles to a room temperature for the metallic nanoparticles to form a doped structure in the light-transmitting substrate, thereby forming the light-triggered light-transmitting cleaning structure.

2. The manufacturing method of claim 1, wherein the light-transmitting substrate is formed of an insulating material.

3. The manufacturing method of claim 2, wherein the light-transmitting substrate is formed of glass.

4. The manufacturing method of claim 3, wherein the metallic nanoparticles are nanometer-sized metal materials or metal oxide materials.

5. The manufacturing method of claim 3, wherein the softening temperature is a softening point temperature of the glass.

6. The manufacturing method of claim 3, wherein the heating time ranges from 3 to 20 minutes.

7. The manufacturing method of claim 2, wherein the light-transmitting substrate is formed of an amorphous polymer.

8. The manufacturing method of claim 7, wherein the metallic nanoparticles are nanometer-sized metal oxide materials.

9. The manufacturing method of claim 7, wherein the softening temperature is between a glass transition temperature (Tg) and a viscous flow temperature (Tf) of the amorphous polymer.

10. The manufacturing method of claim 1, wherein in step S4, the metallic nanoparticles form a grain boundary themselves or with molecules of ambient substances, and the metallic nanoparticles are combined with the grain boundary to form the doped structure.

11. A cleaning method using the light-triggered light-transmitting cleaning structure of claim 10, comprising following steps:
step S5: irradiating the light-triggered light-transmitting cleaning structure with a light source, such that the light source causes a surface plasmon polariton to be formed on a surface of the metallic nanoparticles of the doped structure, and a Tamm plasmon polariton is formed at the grain boundary of the doped structure, whereby the surface plasmon polariton and the Tamm plasmon polariton resonate with each other to form an optical Tamm state; and
step S6: performing an interactive oscillation between the optical Tamm state and the ambient substances of the light-triggered light-transmitting cleaning structure to form a cleaning substance, which spreads outward from a periphery of the light-triggered light-transmitting cleaning structure to remove a pollutant around the light-triggered light-transmitting cleaning structure.

12. The cleaning method of claim 11, wherein a wavelength of the light source ranges from 100 to 1000 nanometers.

13. The cleaning method of claim 11, wherein when the light-transmitting substrate is formed of glass, the wavelength of the light source ranges from 320 to 570 nanometers.
